(19) United States
(12) Patent Application Publication
(10) Pub. No.: US 2015/0257720 A1
(43) Pub. Date: Sep. 17, 2015

(54) METHOD AND SYSTEM FOR AUTOMATICALLY POSITIONING A STRUCTURE WITHIN A FIELD OF VIEW

(71) Applicant: General Electric Company, Schenectady, NY (US)

(72) Inventors: Gil Kovalski, Tirat Carmel (IL); Uri Sneh, Tirat Carmel (IL)

(21) Appl. No.: 14/722,696

(22) Filed: May 27, 2015

Related U.S. Application Data
(63) Continuation of application No. 13/297,507, filed on Nov. 16, 2011.

(51) Int. Cl. A61B 6/04; A61B 6/00; A61B 6/03

(52) U.S. Cl. CPC A61B 6/0407; A61B 6/037; A61B 6/5217; A61B 6/547

(57) ABSTRACT

A method for positioning an organ of interest within a field of view of an imaging detector includes positioning an organ of interest at an initial imaging position, performing automatic organ detection to determine a position of the organ, determining a revised imaging position of a detector or a table based on the position of the organ, prompting a user to accept the revised imaging position, and automatically repositioning at least one of the detector or the table to the revised imaging position based on a user input in response to the prompting. A patient positioning module and a medical imaging system are also described herein.

### RELATED APPLICATIONS

This application is a continuation of U.S. application Ser. No. 13/297,507 entitled “Method and System for Automatically Positioning a Structure Within a Field of View,” filed Nov. 16, 2011, which is hereby incorporated by reference in its entirety.

### BACKGROUND OF THE INVENTION

The subject matter disclosed herein relates generally to nuclear medicine (NM) imaging systems, and more particularly, to a method and system for automatically positioning a structure within the field of view of a NM imaging system.

NM imaging systems, for example, a Single Photon Emission Computed Tomography (SPECT) and a Positron Emission Tomography (PET) imaging system, use one or more imaging detectors to acquire image data, such as gamma ray or photon image data. The image detectors may be gamma cameras that acquire two-dimensional views of three-dimensional distributions of emitted radionuclides (from an injected radioisotope) from a patient being imaged.

When imaging a specific structure, organ or anatomy of the patient, such as the heart, liver or kidney, the patient must be positioned in relation to the detector or camera of the imaging system such that the structure to be imaged is within the field of view (FOV) of one or more of the imaging detectors. If the patient is not positioned correctly, the scan must be stopped and the patient repositioned. In other cases, the positioning problem may not be apparent during the acquisition, and thus acquired data may be reviewed and/or processed before it is found to be deficient. In some cases, incorrect positioning may cause image artifacts such as truncation or distortion of the organ of interest.

Within NM, the patient is typically positioned by an operator who manually adjusts the patient table and the imaging detector(s) while viewing a persistence image until the operator determines that the patient's heart or other structure of interest is centered within the FOV of the detector(s). This may be cumbersome and time consuming depending upon the location of the monitor displaying the persistence image, as well as adding to the discomfort of the patient who needs to lie still on the patient table during the positioning. Long setup time reduces the throughput of the imaging unit, thus reducing the financial gain to the imaging service provider. Manually positioning the patient becomes increasingly complex with NM imaging systems that utilize a plurality small FOV cameras such as a plurality of multi-bores cameras, or a plurality of pinhole cameras, or a pinhole collimator, because the pinhole camera FOV is relatively small. As a result, if the heart is partially outside the FOV, the resultant images may include image artifacts. Accordingly, it may take additional time to properly align the heart within the FOV of the pinhole cameras. Moreover, in a second follow-up examination, the patient must again be positioned on the patient table and the above described procedure repeated to position the organ within the FOV of the imaging detectors to maintain consistency between the two examinations because the evaluation is performed by comparing both images acquired from both scans. Accordingly, each examination of the patient requires the patient to be properly positioned within the image system, thus increasing the overall time required to perform an initial examination and any subsequent follow-up examinations.

### BRIEF DESCRIPTION OF THE INVENTION

In one embodiment, a method for positioning an organ of interest within a field of view of an imaging detector is provided. The method includes positioning an organ of interest at an initial imaging position, performing automatic organ detection to determine a position of the organ, determining a revised imaging position of a detector or a table based on the position of the organ, prompting a user to accept the revised imaging position, and automatically repositioning at least one of the detector or the table to the revised imaging position based on the response. A patient positioning module and a medical imaging system are also described.

In another embodiment, a method of performing at series of medical examinations is provided. The method includes positioning a patient on the table at a first time, performing an initial medical examination of the organ of interest at the revised imaging position at the first time, repositioning the patient on the table at a second different time, calculating a difference between a location of the organ of interest at the first time and the organ of interest at the second different time, and automatically repositioning at least one of the table or the detector based on the calculated difference to perform a second medical imaging examination.

In a further embodiment, a medical imaging system is provided. The medical imaging system includes a detector having a plurality of pinhole cameras, a table, and a patient positioning module for controlling the operation of the detector and the table. The patient positioning module is configured to perform automatic organ detection to determine a position of an organ of interest at a first time and at a first imaging position, determine a revised imaging position of a detector or a table based on the position of the organ, prompt a user to accept the revised imaging position, and automatically reposition at least one of the detector or the table to the revised imaging position based on the response.

### BRIEF DESCRIPTION OF THE DRAWINGS

FIG. 1 is a simplified block diagram of an exemplary imaging system formed in accordance with various embodiments.

FIG. 2 is a flowchart of a method for positioning a structure of interest within a field of view of an imaging detector in accordance with various embodiments.

FIG. 3 is a plurality of persistence images that may be generated in accordance with various embodiments.

FIG. 4 is a plurality of reconstructed images that may be generated in accordance with various embodiments.

FIG. 5 is a plurality of persistence images that may be generated in accordance with various embodiments.

FIG. 6 is a plurality of reconstructed images that may be generated in accordance with various embodiments.

FIG. 7 is a plurality of persistence images that may be generated in accordance with various embodiments.

FIG. 8 is a plurality of reconstructed images that may be generated in accordance with various embodiments.

FIGS. 9A and 9B together depict a flowchart of a method for imaging a patient in accordance with various embodiments.

FIG. 10 is a plurality of persistence images that may be generated in accordance with various embodiments.

FIG. 11 is a plurality of reconstructed images that may be generated in accordance with various embodiments.

FIG. 12 is a plurality of reconstructed images that may be generated in accordance with various embodiments.

FIG. 13 is a plurality of reconstructed images that may be generated in accordance with various embodiments.

FIG. 14 is a perspective view of a Nuclear Medicine (NM) imaging system formed in accordance with various embodiments.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the phrase “reconstructing an image” is not intended to exclude embodiments in which data representing an image is generated, but a viewable image is not. Therefore, as used herein the term “image” broadly refers to both viewable images and data representing a viewable image. However, many embodiments generate, or are configured to generate, at least one viewable image.

Described herein are exemplary imaging systems and methods that enable an operator to automatically and/or semi-automatically position a portion of an organ or structure of interest within a field of view (FOV) of an imaging detector. In various embodiments, the imaging system includes an algorithm that receives information from a plurality of pinhole cameras. The algorithm then processes the received information to generate a visual indication that indicates the current position of the organ of interest. The algorithm also provides a visual indication that represents a suggested position that will place the organ of interest within the FOV of the detector.

The various embodiments are described herein as implemented in connection with a nuclear medicine (NM) imaging system. However, it should be appreciated that although the various embodiments are described in connection with a Single Photon Emission Computed Tomography (SPECT) imaging system having a particular configuration, the various embodiments may also be implemented in connection with a Positron Emission Tomography (PET) imaging system, as well as with other types of single or dual-modality imaging systems.

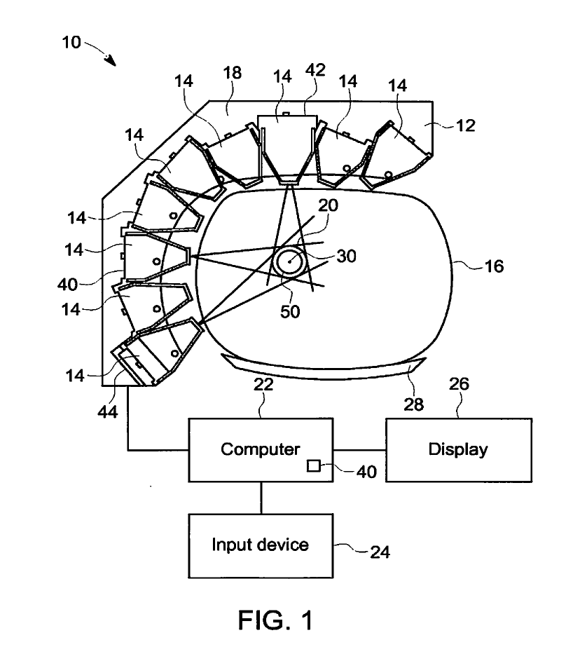

FIG. 1 is a simplified block schematic diagram of a portion of an exemplary imaging system 10 formed in accordance with various embodiments. The imaging system 10 includes an NM camera configured as a SPECT detector 12. The SPECT detector 12 includes N detector modules 14. In one embodiment, N=19. In some embodiments, the array of modules 14 is a multidimensional array wherein modules 14 are arranged in a plurality of rows (only one such row is seen in the cross-sectional view depicted in FIG. 1). It should be noted that the various embodiments are not limited to the NM imaging system 10 having a single detector 12 operable to perform SPECT imaging. For example, the NM imaging system 10 may include one or more additional detectors 12 such that the detectors 12 are provided having a central opening there through.

In one embodiment, the detector modules 14 are formed from pixelated detector elements that may operate, for example, in an event counting mode and may be configured to acquire SPECT image data. The detector modules 14 may be formed from different materials, particularly semiconductor materials, such as cadmium zinc telluride (CZT), cadmium telluride (CdTe), and silicon (Si), among others. In various embodiments, the plurality of detector modules 14 each include detector elements having a plurality of pixels. However, it should be noted that the various embodiments are not limited to a particular type or configuration of detectors, and any suitable imaging detector may be used.

The detector modules 14 may include pinhole collimation formed from pinhole collimators that may be coupled to the face of the detector modules 14. The collimators, in some embodiments, define a multi-pinhole collimator arrangement. As used herein, the term “pinhole camera” refers to the combination of a single detector module 14 and a corresponding collimator, if utilized. Accordingly, in the exemplary embodiment, the detector 12 is formed to include N pinhole cameras 14.

In the illustrated embodiment, the pinhole cameras 14 are arranged and supported on a support structure 18 (e.g., a scanner gantry) in a generally curved or arcuate configuration. Thus, the detector 12 has a generally semi-arc shape or L-shaped arrangement similar to an L-mode of operation. The pinhole cameras 14 may be arranged to provide, for example, organ specific imaging such that each of the pinhole cameras 14 is fixed on the support structure 18 and conforms to the shape of the patient 16. However, the pinhole cameras 14 may be configured for any type of organ specific imaging or for general purpose imaging.

In the exemplary embodiment, the pinhole cameras 14 are arranged on the support structure 18 such that the field of view for each pinhole camera 14 is focused on the same region of interest (ROI). For example, as shown in FIG. 1, the pinhole cameras 14 are each aligned to receive information from a single ROI 20. Thus the FOV's for each respective pinhole camera 14 overlap to encompass the ROI 20.

The imaging system 10 also includes a computer 22. The computer 22 is programmed to perform functions described herein, and as used herein, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein.

The imaging system 10 also includes an input device 24 that is configured to receive information from a user and transmit information to the computer 22. The imaging system 10 further includes a display device 26 for displaying various images as described in more detail below.

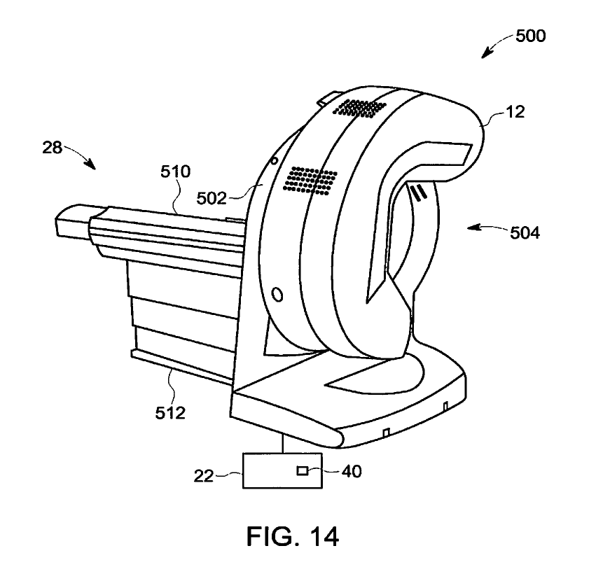

The imaging system 10 further includes a table 28 having a bed 510 (shown in FIG. 14), for supporting the patient 16 during the examination procedure. The table 28 is configured to facilitate ingress and egress of the patient 16 into an examination position. In some embodiments, table 28 comprises motion controllers such as computer-controlled electrical motors and position encoders to facilitate the positioning of table 510. In operation, and generally prior to the imaging system 10 being utilized to perform an examination, the imaging system 10 is calibrated or aligned such that the location of the bed 510 with respect to the location of the detector 12 is known. For example, as discussed above, the detector 12 includes a plurality of pinhole cameras 14. In the exemplary embodiment, the pinhole cameras 14 are each aligned such that a centerline of the FOV of each pinhole camera 14 intersects at a predetermined focal point 30, as shown in FIG. 1. Accordingly, the imaging system 10 has a priori information of the x, y, and z coordinates of the focal point 30 in three-dimensional (3D) space. Moreover, the imaging system 10 also includes a priori information on the location of the bed 510 in 3D space. For example, the imaging system 10 has a priori information that describes the x, y, and z axis location of the table 28. Accordingly, the imaging system 10 has a priori information that describes the imaging system geometry and in particular describes the 3D coordinate positions of both the detector 12 and the bed 510.

The imaging system 10 also includes a patient positioning module 40 that is configured to implement various methods and algorithms described herein. The patient positioning module 40 may be implemented as a piece of hardware that is installed in the computer 22. Optionally, the patient positioning module 40 may be implemented as a set of instructions that are installed on the computer 22. The set of instructions may be stand alone programs, may be incorporated as subroutines in an operating system installed on the computer 22, may be functions in an installed software package on the computer 22, and the like. It should be understood that the various embodiments are not limited to the arrangements and instrumentality shown in the drawings. Accordingly, in the exemplary embodiment, the patient positioning module 40 includes a set of instructions, such as for example, an algorithm that enables the computer 22 to perform the various methods described herein.

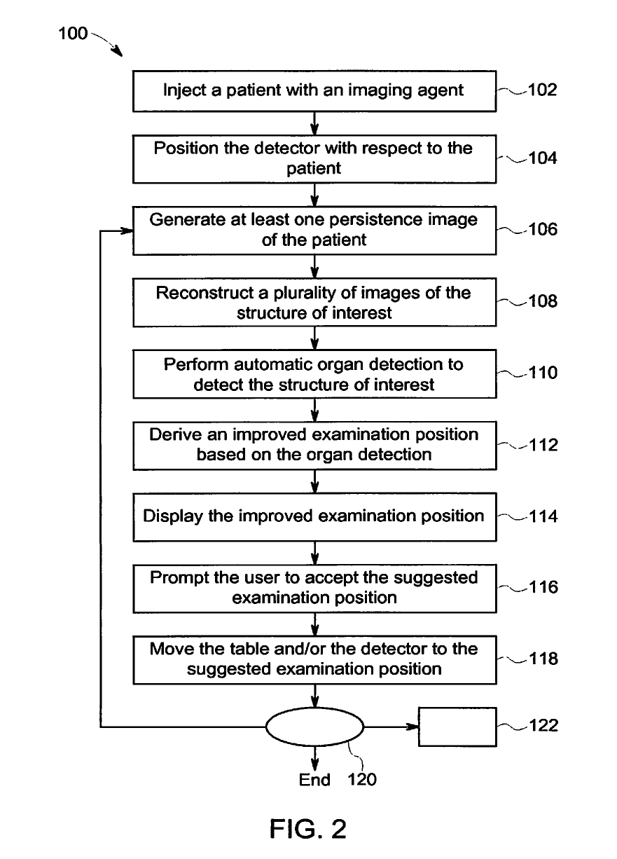

FIG. 2 is a flowchart illustrating an exemplary method 100 for positioning a structure of interest within a field of view of an imaging detector, such as the detector 12. The method 100 may be implemented using the patient positioning module 40 described above.

At 102, a patient, such as for example the patient 16 is injected with an imaging agent. The term “imaging agent,” as used herein includes any and all radiopharmaceutical (RP) agents and contrast agents used in connection with diagnostic imaging and/or therapeutic procedures. The imaging agent may represent a perfusion agent. The imaging agent may be, among other things, an imaging agent adapted for use in an NM system, such as NM system 10. By way of example only, the imaging agent may be Myoview™, Fluorodeoxyglucose (FDG), ¹⁸F-Flourobenzyl Triphenyl Phosphonium (¹⁸F-FBnTP), ¹⁸F-Flouroacetate, ¹⁸F-labeled myocardial perfusion tracers, Tc-ECD, Tc-HMPAO, N-13 ammonia, Envision N-13H3, Iodine-123 ligands, ^{99m}-Technitium ligands, Xenon-133, Neuroreceptor ligands, etc.), ¹⁸F-fluoromisonidazole, ²⁰¹Thallium, ^{99m}Technetium sestamibi, and ⁸²Rubidium, among others.

In various embodiments, the imaging agent is selected based on the particular type of study desired. For example, a technetium ligand imaging agent or a thallium imaging agent may be utilized during a heart study. It should be realized that although various embodiments are described herein with respect to performing a heart study, that the methods and algorithms described herein may be utilized to image any organ or structure within the patient 16. Accordingly, the imaging agent injected into the patient 16 is selected based on the type of study being implemented.

At 104, the detector 12 is positioned with respect to the organ of interest. In various embodiments, the bed 510, and therefore the organ of interest, may be moved along an x, y, and/or z axis with respect to the detector 12. In some embodiments, detector 12 may rotate about the bore 504 in the gantry (see FIG. 14). In various other embodiments, the detector 12 may be moved along an x, y, or z axis to image the organ of interest. Optionally, both the table 28 and the detector 12 are moved to perform the examination. As described above, the imaging system 10 has a priori information that describes the imaging system geometry and in particular describes the 3D coordinate positions of both the detector 12 and the bed 510.

In operation, a user may depress a button on the input device 24 to activate the examination procedure. Optionally, the user may depress an icon on the display device 26 to activate the examination procedure. Activating the examination procedure causes the table 28, and therefore the organ of interest, to be moved into the FOV of the detector 12. As described above, the position of the table 28 and the position of the detector 12 are known, accordingly, the system 10 is capable of determining an initial position of both the detector 12 and the table 28. In the exemplary embodiment, when the examination is initiated, the detector 12 and/or the table 28 are moved from the initial position to a first examination position. In the first examination position, the organ of interest is positioned in relation to the detector 12 such that the organ of interest, e.g. the heart, is at least partially within the field of view (FOV) of one or more of the pinhole cameras 14.

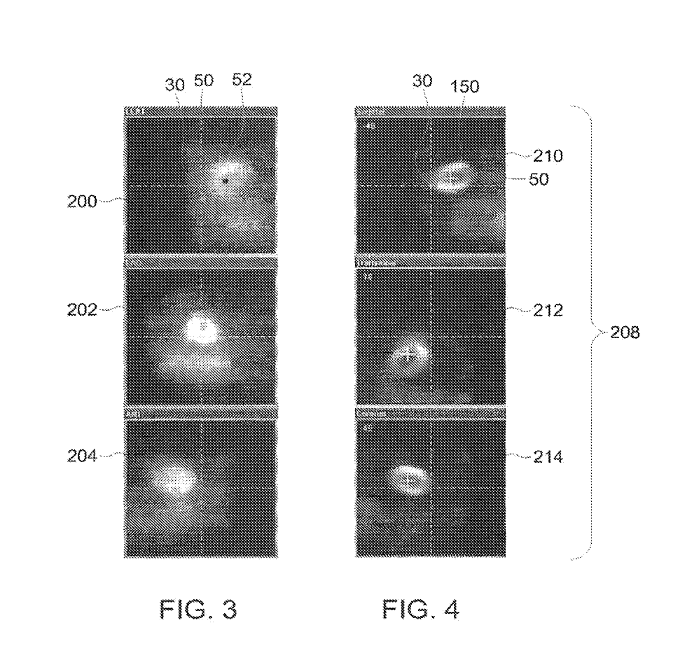

At 106, at least one persistence image is generated and displayed in real time. A persistence image is an image that depicts the decay of the imaging agent injected into the patient 16, and is not a reconstructed image. Accordingly, a plurality of persistence images depict the decay of the imaging agent injected over the previous few seconds from different angles which are acquired by different pinhole cameras 14. For example, FIG. 3 illustrates three exemplary persistence images 200, 202, and 204 that may be acquired at 106. While the exemplary embodiment is described with respect to three persistence images, it should be realized that up to N persistence images may be generated. More specifically, the information acquired from each pinhole camera 14 may be utilized to generate a respective persistence image. In the exemplary embodiment, the methods described herein are utilized to generate three persistence images 200, 202, and 204 that are acquired from three different imaging angles by three different pinhole cameras 14. For example, as shown in FIG. 3, in the various embodiments, a pinhole camera 40 (shown in FIG. 1) may be utilized to acquire the persistence image 200 (shown in FIG. 3), a pinhole camera 42 (shown in FIG. 1) may be utilized to acquire the persistence image 202 (shown in FIG. 3), and a pinhole camera 44 (shown in FIG. 1) may be utilized to acquire the persistence image 204 (shown in FIG. 3). In the exemplary embodiment, the persistence images 200, 202, and 204 are displayed to the operator in real-time. The dashed crosshairs in each of the persistence images 200, 202, and 204 represent the center of the FOV of the pinhole cameras 14. More specifically, the crosshairs represent the focal point 30 of the pinhole cameras 14.

At 108, the information acquired from the pinhole cameras 14 is utilized to reconstruct a 3D reconstruction of the ROI 20 which includes at least parts of the organ of interest. In an exemplary embodiment, the 3D reconstruction of the ROI 20 is used for generating reconstructed slice images. In other exemplary embodiments, the 3D reconstruction of the ROI 20 is used for generating of reconstructed projection images. Reconstructed projection images are representations of what a specific pinhole camera would detect if it was presented with the 3D reconstruction of the ROI 20. The reconstructed projection images are generated taking into account the relative positions of the reconstructed ROI and the pinhole camera, the physical properties of the camera, and optionally other influences such as attenuation. In various embodiments, the organ of interest is a heart 50 (shown in FIG. 1). In operation, the information acquired from the pinhole cameras 14 may be utilized by the computer 22 to generate a set 208 of reconstructed images. FIG. 4 illustrates an exemplary set 208 of reconstructed images that may include, for example, a sagittal image 210, a transaxial image 212, and/or a coronal image 214. However, it should be realized that set 208 of reconstructed images may include more than three reconstructed images or less than three reconstructed images and may be from different angles or views. The reconstructed images 210, 212, and 214 may be displayed with the persistence images 200, 202, and 204 to enable the user to view both sets of images concurrently. The dashed crosshairs shown in the reconstructed images 210, 212, and 214 represent the center of the FOV of the pinhole cameras 14. More specifically, the crosshairs represent the focal point 30 of the pinhole cameras 14.

At 110, automatic organ detection is performed using the set 208 of reconstructed images. More specifically, the method 100 enables fully automatic detection of the organ of interest, such as for example, the heart 50. While various embodiments are described with respect to automatically detecting the heart 50, it should be realized that other structures and/or organs of interest may be automatically detected based on the user input.

In the exemplary embodiment, to automatically detect the heart 50, the set 208 of reconstructed images is accessed. In operation, the patient positioning module 40 is programmed to include a priori information of at least one known heart (not shown). Such a priori information may include, for example, expected uptake times and locations when the uptake is expected to be in the heart region. Other a priori information may include expected heart pixel intensity values based on various heart studies that have been previously performed that include pixel intensity values that represent known hearts. Thus, assuming that the patient positioning module 40 has information of pixel intensity values that more than likely represent pixels of the known heart, the patient positioning module 40 may utilize this information to locate the heart 50 in at least one image in the set 208 of reconstructed images. In the exemplary embodiment, the patient positioning module 40 may be trained using a large set of known hearts to generate a heart training dataset. In operation, the training dataset may include information is based on a shape of a plurality of known hearts. Thus, the outline or shape of the heart 50 in the set 208 of reconstructed images may be calculated and then compared to the outline or shape of a plurality of known hearts to determine whether the region suspected to define the heart 50 does in fact define the heart 50.

In other embodiments, the heart 50 may be identified by comparing objects within one of the reconstructed images in the set 208 of reconstructed images to other objects within the same reconstructed image. For example, it is known that the heart 50 is located in the chest cavity proximate to the lungs, the spine, the liver, etc. Thus, the patient positioning module 40 may determine a shape and/or size of various organs or objects within the chest cavity to identify the heart 50. Accordingly, at 110, the patient positioning module 40 is configured to identify the location of the heart 50 and more specifically define a point 52 (shown in FIG. 1) that represents a center of the heart 50. The patient positioning module 40 is further configured to automatically place a visual indication 150 on the reconstructed images 210, 212, and/or 214 that represent the center 52 of heart 50. In various embodiments, the visual indication 150 is a set of crosshairs having a predetermined color. In one embodiment, the visual indication 150 is yellow. Optionally, the visual indication 150 may be colors other than yellow.

At 112, the location of the focal point 30 is compared to the location of the heart center point 52, which is shown by the visual indication 150, to derive an improved examination position. For example, as described above, when the examination is initiated, the detector 12 and/or the table 28 are moved from the initial position to the first examination position. In the first examination position, the patient 16 is positioned in relation to the detector 12 such that the organ of interest, e.g. the heart 50, is at least partially within the field of view (FOV) of one or more of the pinhole cameras 14. However, as shown in FIG. 3, in at least some reconstructed images 208, the center of the heart 52, shown by the visual indication 150, is not centered within the ROI 20. More specifically, the center of the heart 52 is located at a different position than the focal point 30 of the pinhole cameras 14. Thus, the heart 50 in the first examination position is not positioned at an optimal imaging position. The term “optimal imaging position” as used herein means that the center of the heart 52 is located proximate to the focal point 30 of the pinhole cameras 14. Accordingly, at 112, the patient positioning module 40 is configured to calculate a revised table position and/or a revised detector position that would enable the center of the heart 52 to be located proximate to, and/or overlaid onto, the focal point 30 of the pinhole cameras 14. It should be noted that the term “focal point” and “center of the organ” are to be viewed broadly as the ROI and/or the organ of interest may not have a symmetrical shape.

At 114, the results indicating the revised detector 12 and/or table 28 position may be displayed to the user on, for example, the display 26. The results may be displayed as numerical values that indicate, for example, the current location of the detector 12 and/or the table 28 and/or the suggested location of the detector 12 and/or the table 28. For example, assuming that the current axial location of the table 28 is zero centimeters (cm), the patient positioning module 40 may suggest that the table 28 be moved axially into the gantry to a table position of 38.4 cm. Additionally, the patient positioning module 40 may also suggest that the detector 12 be moved radially inward to a position of 19 cm. It should be realized that the above examples are only exemplary, and that the patient positioning module 40 is configured to suggest movement of the detector 12 and/or the table 28 to any position that enables the center of the heart 52 to be located proximate to, or overlaid with, the focal point 30 of the pinhole cameras 14.

At 116, the user is prompted to accept the suggested examination position. To accept the suggested examination position, the user may depress a button or icon to enable the detector 12 and/or the table 28 to be automatically moved to the suggested position also referred to herein as the second examination position. Optionally, the user may manually move the detector 12 and/or the table 28 to the suggested position. The term “manually” as used herein means that the user may operate various devices, such as for example, a table controller or a detector controller, to cause the detector 12 and/or the table 28 to be moved to the suggested position. The term “manually” also means that the operator may physically reposition the detector 12 and/or the table 28 without the use of the table controller or the detector controller.

At 118, the detector 12 and/or the table 28 are moved to the suggested examination position, i.e. the second examination position, either automatically or manually as described above. Additionally, the results indicating the revised position are displayed to the user on, for example, the display 26. The results may be displayed as numerical values that indicate, for example, the current location of the detector 12 and/or the table 28 after the suggested position has been implemented. After the detector 12 and/or the table 28 are moved to the second examination position, an additional set of persistence images is generated. Moreover, a second set of reconstructed images may be generated. Optionally, safety measures are taken to avoid collision of the patient with the camera parts. Such measures may be proximity and/or contact sensors installed on the camera. Optionally, the operator is required to hold down a switch and view the patient during the motorized motion to the new position.

At 120, the user determines if the second examination position is acceptable to perform a medical imaging scan of the patient 16. In general, the user may look at the set of reconstructed images 228 to make the determination. If the user determines that the detector 12 and the table 29 are located in an acceptable imaging position, the method 100 is completed.

However, in various embodiments, the user may determine that the center of the heart 52 is not aligned with the focal point. In this embodiment, at 120, the user may manually select or enter a command that directs the patient positioning module 40 to repeat steps 106-118. In various embodiments, at 120, new persistent images may be acquired and used to determine if the center of the heart 52 is aligned with the focal point. Optionally, new of set of reconstructed images 228 to make the determination.

In various other embodiments, when the center of the heart 52 is not located at a position in the FOV that is satisfactory to the user, at 122, the user may manually place a pointer on the visual indication 160 and move the visual indication 160 to a desired location. In response, the patient positioning module 40 is configured to automatically determine the coordinates of the revised table position and/or a revised detector position based on the input received by the user. The results indicating the revised detector 12 and/or table 28 positions may be displayed to the user on, for example, the display 26 as described above. The user may then be prompted to move the detector 12 and/or the table 28 automatically or the user may manually move the detector 12 and/or the table 28 to the revised position.

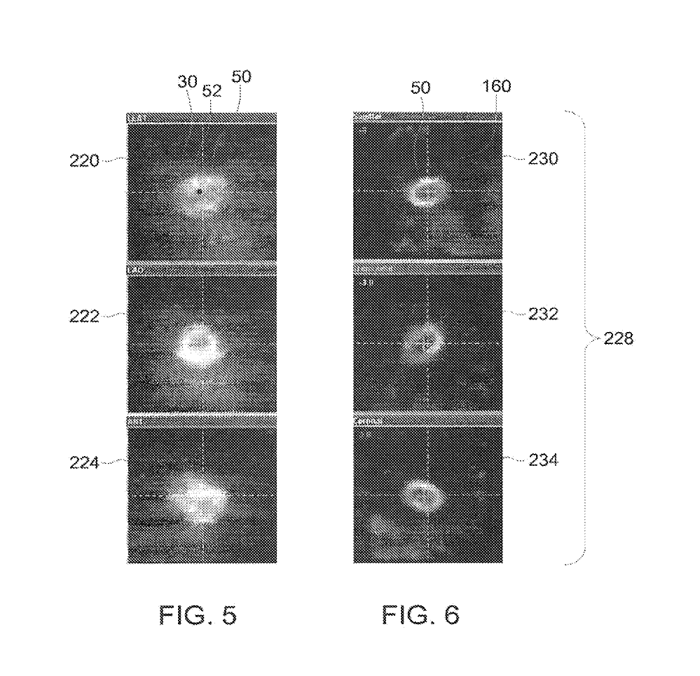

A second set of persistence images may then be acquired. For example, FIG. 5 illustrates three exemplary persistence images 220, 222, and 224 that may be acquired at the second examination position. In the exemplary embodiment, the persistence images 220, 222, and 224 are displayed to the operator in real-time. Moreover, the dashed crosshairs in each of the persistence images 220, 222, and 224 represent the center of the FOV of the pinhole cameras 14. More specifically, the crosshairs represent the focal point 30 of the pinhole cameras 14. Similarly, information acquired from the pinhole cameras 14, at the second examination position, may be utilized by the computer 22 to generate a set of reconstructed images. FIG. 6 illustrates an exemplary set 228 of reconstructed images that may include, for example, a sagittal image 230, a transaxial image 232, and/or a coronal image 234. However, it should be realized that set 228 of reconstructed images may include more than three reconstructed images or less than three reconstructed images. The reconstructed images 230, 232, and 234 may be displayed with the persistence images 220, 222, and 224 to enable the user to view both sets of images concurrently. The dashed crosshairs shown in the reconstructed images 230, 232, and 234 represent the center of the FOV of the pinhole cameras 14. More specifically, the crosshairs represent the focal point 30 of the pinhole cameras 14.

The patient positioning module 40 is then configured to automatically place a visual indication 160 on the reconstructed images 230, 232, and/or 234 that represent the center of heart 50 at revised position, e.g. the position suggested by the user. In various embodiments, the visual indication 160 is a set of crosshairs having a predetermined color that is different than the color of the visual indication 150. In the exemplary embodiment, the visual indication 160 is green to indicate that the user has provided a location to move the detector 12 and/or the table 14 to align the center of the heart 58 with the focal point 30. Optionally, the visual indication 160 may be colors other than green but different than the color of the visual indication 150.

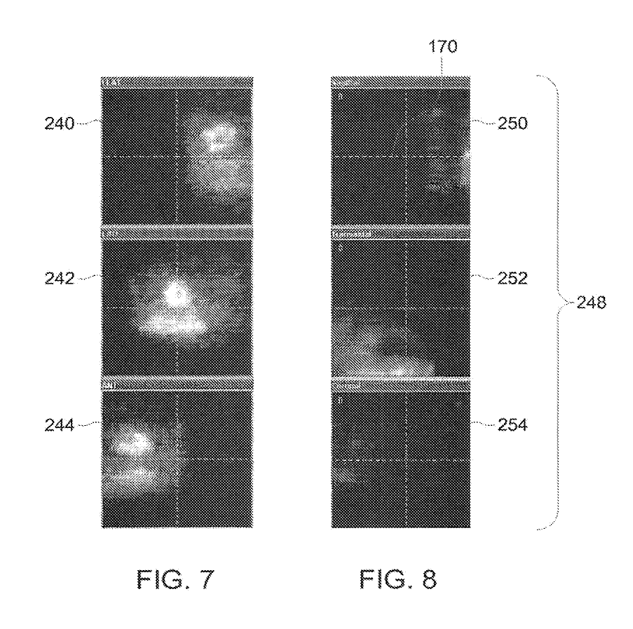

In various other embodiments, the patient positioning module 40 may not properly perform organ detection at 108. In this case the center of the heart 152 is not properly positioned with respect to the focal point 30. For example, FIG. 7 illustrates three exemplary persistence images 240, 242, and 244 that may be acquired at the second examination position. In the exemplary embodiment, the persistence images 240, 242, and 244 are displayed to the operator in real-time. Moreover, the dashed crosshairs in each of the persistence images 240, 242, and 244 represent the center of the FOV of the pinhole cameras 14. Similarly, information acquired from the pinhole cameras 14, at the second examination position, may be utilized by the computer 22 to generate a set of reconstructed images. FIG. 8 illustrates an exemplary set 248 of reconstructed images that may include, for example, a sagittal image 250, a transaxial image 252, and/or a coronal image 254. However, it should be realized that set 248 of reconstructed images may include more than three reconstructed images or less than three reconstructed images. The reconstructed images 250, 252, and 254 may be displayed with the persistence images 240, 242, and 244 to enable the user to view both sets of images concurrently. The dashed crosshairs shown in the reconstructed images 250, 252, and 254 represent the center of the FOV of the pinhole cameras 14.

Accordingly, at 122, the patient positioning module 40 is configured to automatically place a visual indication 170 on the reconstructed images 250, 252, and/or 254 at the location of the focal point 30 when the heart has not been automatically detected at 108. In various embodiments, the visual indication 170 is a set of crosshairs having a predetermined color that is different than the color of the visual indication 150 and the visual indication 160. In the exemplary embodiment, the visual indication 170 is red to indicate that patient positioning module 40 failed to detect the heart 50. Optionally, the visual indication 170 may be any color that is different than the color of the visual indication 150 and the visual indication 160. The user may then observe the persistence images, which are generated in real time, while manually moving the detector 12 and or the table 28 until the heart 50 is positioned proximate to the focal point 30. The method 100 may then be repeated.

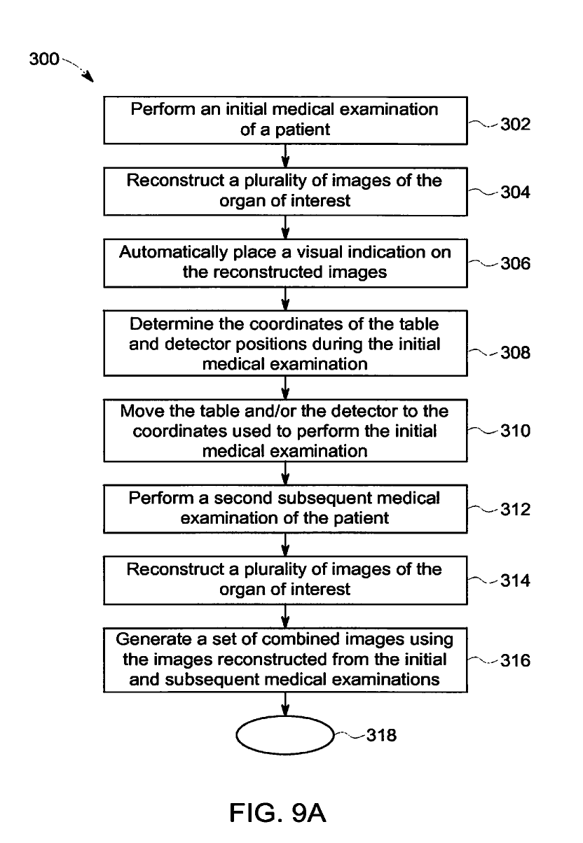

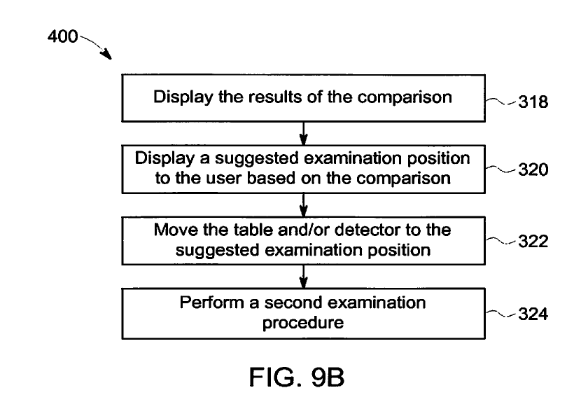

FIGS. 9A and 9B are a flowchart illustrating an exemplary method 300 of aligning the patient for a second medical imaging examination. In various embodiments, to perform a NM examination, the patient 16 is examined a plurality of times. For example, example, a patient may undergo a second examination as a follow-up after treatment such as chemotherapy or catheterization. In another example, a cardiac patient may be examined in a stress condition and a rest condition. For example, the user may instruct the patient 16 to walk, jog, and/or run for a predetermined amount of time prior to being imaged to place the heart 50 in a stress condition. The stress condition may also be chemically induced. Performing medical imaging examinations while the heart is in a stress condition and a rest condition enables the user to more clearly identify various diseases or abnormalities, such as for example, heart defects. Accordingly, if the images of the heart 50 in the stress condition and the rest condition are substantially similar, this may provide an indication that the heart 50 is functioning properly. However, if the images of the heart 50 in the stress condition are different than the images of the heart 50 in the rest condition, this may provide an indication that the heart 50 is functioning improperly or has some defect. In a repeated imaging, consistent patient positioning may be important for repayable comparison between the two images. It should be noted that internal organs within the patient may shift or move between imaging sessions. For example, the heart may move due to the abovementioned stressing and slowly return to its resting position.

Accordingly, at 302, the system 10 is configured to perform an initial medical examination. In the exemplary embodiment, the patient 16 is injected with a first imaging agent having a first energy level to prior to performing the initial medical examination. To perform the initial medical examination, the detector 12 and/or the table 28 may be positioned as described above in the method 100. After the heart 50 is properly positioned for scanning, the initial medical examination of the patient 16 is performed. In one embodiment, the patient 16 may be in the stress condition. Optionally, the patient 16 may be in the rest condition. The set of medical examination images have a quality to enable the user to perform a medical diagnosis of the patient 16.

At 304, a set of coordinates that indicate the position of the detector 12 and the table 28 used to perform the initial medical examination at the first time are uploaded to the patient positioning module 40.

At 306, the detector 12 and/or the table 28 are repositioned to the same position utilized to perform the initial medical examination. More specifically, the patient positioning module 40 is configured to utilize the coordinates accessed at 308 to prompt the operator to either automatically or manually reposition the detector 12 and/or the table 28 to the same coordinates utilized to perform the initial medical examination.

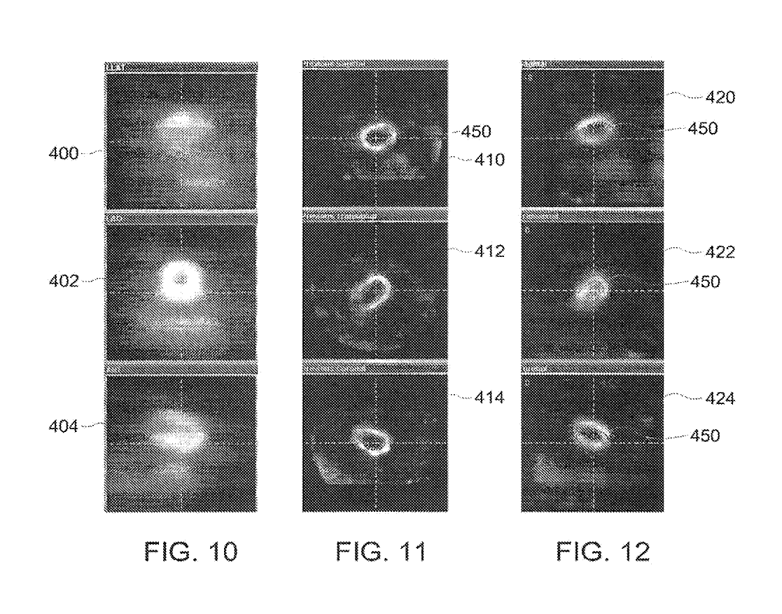

At 308, an initial set of persistence images are acquired while the detector 12 and the table 28 are positioned at the same coordinates utilized during the first medical examination. For example, FIG. 10 illustrates three exemplary persistence images 400, 402, and 404 that may be acquired at 302. The dashed crosshairs in each of the persistence images 400, 402, and 404 represent the center of the FOV of the pinhole cameras 14. More specifically, the crosshairs represent the focal point 30 of the pinhole cameras 14.

In one embodiment, the patient 16 may be injected with the same imaging agent used to generate the initial set of persistence images. In the exemplary embodiment, the patient 16 is injected with a second imaging agent, having a second energy level that is different than the first imaging agent, having the first energy level that is used to perform the first medical examination. Moreover, in the exemplary embodiment, the second follow-up medical examination is performed at a different time than the first medical examination. Additionally, if the initial medical examination is performed under a stress condition, the second follow-up examination is performed under a rest condition. Optionally, if the initial medical examination is performed under a rest condition, the second follow-up examination is performed under a stress condition. The second medical examination may also be performed using the method 100 described above. For example, the second medical examination may be performed two hours after the first medical examination, the next day, etc.

At 310, the patient positioning module 40 is configured to instruct the processor to reconstruct a plurality of images. For example, FIG. 11 illustrates three reconstructed images 410, 412, and 414 that may be reconstructed. In various embodiments, the reconstructed images 410, 412, and 414 may be displayed with the persistence images 400, 402, and 404 to enable the user to view both sets of images concurrently. The dashed crosshairs shown in the reconstructed images 410, 412, and 414 represent the center of the FOV of the pinhole cameras 14. More specifically, the crosshairs represent the focal point 30 of the pinhole cameras 14.

At 312, the patient positioning module 40 is further configured to automatically place a visual indication 450 on the reconstructed images 410, 412, and/or 414 that represent an outline of the heart 50. Optionally, the visual indication 450 may be positioned to indicate the center of the heart 52. In various embodiments, the visual indication 450 has a shape that is selected to conform to the myocardium of the heart 50. Such indication 450 having a shape that is selected to conform to the myocardium of the heart 50 may be generated for example by using a threshold value to locate the myocardium in the reconstructed image. In the exemplary embodiment, the visual indication 450 is red. Optionally, the visual indication 450 may be colors other than red.

At 314, the patient positioning module 40 is configured to generate a set of combined images using the images reconstructed from the initial medical examination and the images reconstructed at 310. For example, FIG. 12 illustrates a combined image 420, a combined image 422, and a combined image 424. Additionally, the combined images 420, 422, and 424 include the visual indication 450.

In various embodiments, the heart 50 may not be properly located in the detector FOV during the second medical examination even though the detector 12 and the table 28 are positioned in the same locations as used to perform the medical imaging in the first medical examination. The patient 16 may be located at a different position on the bed 510 and/or the heart 50 may be in a rest or stress condition, thus the heart 50 may be in a different location in the chest cavity. For example, referring again to image 420 in FIG. 12, the visual indication 450 represents the position of the heart 50 during the initial medical examination which is not aligned with the image of the heart acquired during the second follow-up examination. Similarly, the visual indication 450 in image 424 is not aligned with the image of the heart acquired during the second follow-up examination.

Accordingly, at 316, the location of the visual indication 450, which indicates the position of the heart 50 during the initial medical examination, is compared to the location of the heart 50 in the second position. More specifically, the patient positioning module is configured to calculate a revised table position and/or a revised detector position that would enable the heart 50 to be located at the same position in the second follow-up examination as the heart 50 was located in the initial medical examination.

At 318, the results indicating the revised detector 12 and/or table 28 position may be displayed to the user on, for example, the display 26. The results may be displayed as numerical values that indicate, for example, the current location of the detector 12 and/or the table 28 and/or the suggested location of the detector 12 and/or the table 28.

At 320, the user is prompted to accept the suggested examination position. To accept the suggested examination position, the user may depress a button or icon to enable the detector 12 and/or the table 28 to be automatically moved to the suggested position also referred to herein as the second examination position. Optionally, the user may manually move the detector 12 and/or the table 28 to the suggested position by selecting a dragging the visual indication 450 to the desired location.

At 322, the detector 12 and/or the table 28 are moved to the suggested examination position, i.e. the second examination position, either automatically or manually as described above. Additionally, the results indicating the revised position are displayed to the user on, for example, the display 26. The results may be displayed as numerical values that indicate, for example, the current location of the detector 12 and/or the table 28 after the suggested position has been implemented.

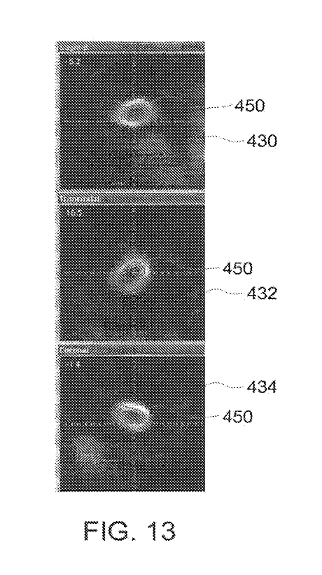

After the detector 12 and/or the table 28 are moved to the second examination position, at 324, the second examination procedure is performed. Moreover, a second set of combined images may be generated. For example, FIG. 13 illustrates a combined image 430, a combined image 432 and a combined image 434. As shown in FIG. 13, after the detector 12 and/or the table 28 have been repositioned, the heart 50 in the first examination position, shown by the visual indication 450, is in substantially the same location in the second medical examination. If the user determines that the detector 12 and the table 29 are located in an acceptable imaging position, the second medical examination is performed. Optionally, the user may choose to repeat the method 300 to further optimize the position of the detector 12 and the table 28.

Various embodiments described herein provide a method and/or system for automatically or semi-automatically positioning a patient to perform a medical imaging examination. Various embodiments provide a method and system for improving image quality by assisting the user in centering the organ of interest within the FOV of a detector, and providing consistent patient positioning during subsequent examinations.

The various embodiments may be implemented in connection with any imaging system. For example, FIG. 14 is a perspective view of an exemplary imaging system 500 that may be utilized to implement the various methods described herein. The system 500 includes a gantry 502 having a gantry central bore 504. The gantry 502 is configured to support one or more NM radiation detectors, which may be configured as CZT imaging modules, for example, the detector 12 (shown in FIG. 1) that is supported, for example, around approximately 180 degrees of the gantry 502. The patient table 28 may include a bed 510 slidingly coupled to a bed support system 512, which may be coupled directly to a floor or may be coupled to the gantry 502 through a base coupled to the gantry 502. The table 28 may include a bed 510 slidingly coupled to an upper surface of the table 28. The patient table 28 is configured to facilitate ingress and egress of a patient (not shown) into an examination position that is substantially aligned with the examination axis of the gantry central bore 502. The patient table 28 may also configured to facilitate up and down motion of bed 510. During an imaging scan, the patient table 28 may be controlled to move the patient 16 axially into and out of (as well as upward and downward within) the gantry central bore 504 to obtain event count information for the patient or a region of the patient. The operation and control of the imaging system 500 may be performed in any manner known in the art. It should be noted that the various embodiments may be implemented in connection with imaging systems that include stationary gantries or moving gantries. Additionally, the imaging system 500 may include the computer 22 and the patient positioning module 40 as described herein.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as an optical disk drive, solid state disk drive (e.g., flash RAM), and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for positioning an organ of interest within a field of view of an imaging detector, the method comprising:
positioning the organ of interest at an initial imaging position;
acquiring initial image data while the organ of interest is in the initial imaging position;
reconstructing an initial image from the initial image data acquired while the organ of interest is in the initial imaging position;
performing automatic organ detection to determine the initial imaging position of the organ of interest;
deriving an improved examination position based, at least in part, on the initial position of the organ of interest;
moving the patient to the improved examination position; and
performing an examination of the organ of interest at the improved imaging position.

2. The method of claim 1, further comprising reconstructing an improved image from the performing an examination operation.

3. The method of claim 1, wherein the deriving operation comprises comparing a first location of a focal point of the imaging detector to a second location of at least a portion of the organ of interest.

4. The method of claim 1, wherein the performing automatic organ detection operation is based on a priori information of the organ of interest.

5. The method of claim 1, wherein the acquiring initial image data comprises acquiring at least one persistence image.

6. The method of claim 1, further comprising repeating each of the positioning the organ of interest operation, the acquiring initial image data operation, the reconstructing an initial image operation, the performing automatic organ detection operation, the deriving operation, and the moving operation before the performing an examination operation.

7. The method of claim 1, further comprising automatically placing a visual indication on at least one reconstructed image.

8. The method of claim 7, further comprising changing a color of the visual indication from a first color to a second color that differs from the first color in response to the imaging detector or a table moving to improved imaging position.

9. The method of claim 7, further comprising changing a color of the visual indication from a first color to a second color that differs from the first color in response to the organ of interest not being detected.

10. The method of claim 1, further comprising:
determining coordinates of a table and the imaging detector during an initial examination;
moving one or both of the table or the imaging detector to the coordinates used to perform the initial examination;
performing a subsequent examination; and
generating a set of combined images using images reconstructed from the initial and subsequent examinations.

11. A medical imaging system comprising:
a detector comprising a plurality of pinhole cameras;
a table; and
at least one processor for controlling the operation of the detector and the table, the at least one processor configured to:
acquire initial image data while an organ of interest is in an initial imaging position;
reconstruct an initial image from the initial image data acquired while the organ of interest is in the initial imaging position;
perform automatic organ detection to determine the initial imaging position of the organ of interest; and
derive an improved examination position based, at least in part, on the initial position of the organ of interest.

12. The medical imaging system of claim 12, wherein the at least one processor is further configured to reconstruct an improved image based on an examination of the organ of interest at the improved imaging position.

13. The medical imaging system of claim 12, wherein the at least one processor is further configured to compare a first location of a focal point of the imaging detector to a second location of at least a portion of the organ of interest to derive the improved examination position.

14. The medical imaging system of claim 12, wherein the at least one processor is further configured to base the automatic organ detection on a priori information of the organ of interest.

15. The medical imaging system of claim 12, wherein the at least one processor is further configured to acquire at least one persistence image.

16. The medical imaging system of claim 12, wherein the at least one processor is further configured to repeat each of the acquire operation, the reconstruct operation, the perform operation, and the derive operation before an examination of the organ of interest.

17. The medical imaging system of claim 12, wherein the at least one processor is further configured to automatically place a visual indication on at least one reconstructed image.

18. The medical imaging system of claim 17, wherein the at least one processor is further configured to change a color of the visual indication from a first color to a second color that differs from the first color in response to the imaging detector or a table moving to improved imaging position.

19. The medical imaging system of claim 17, wherein the at least one processor is further configured to change a color of the visual indication from a first color to a second color that differs from the first color in response to the organ of interest not being detected.

20. The medical imaging system of claim 12, wherein the at least one processor is further configured to:
determine coordinates of a table and the imaging detector during an initial examination;
move one or both of the table or the imaging detector to the coordinates used to perform the initial examination;
perform a subsequent examination; and
generate a set of combined images using images reconstructed from the initial and subsequent examinations.
